(19) United States
(12) Patent Application Publication
(10) Pub. No.: US 2021/0196772 A1
(43) Pub. Date: Jul. 1, 2021

(54) CONFECTIONS FOR SKIN PROTECTION: COMPOSITIONS, METHODS FOR MAKING, AND APPLICATIONS THEREOF

(71) Applicant: Grove Collaborative, Inc., San Francisco, CA (US)

(72) Inventors: Emilia JAVORSKY, Watertown, MA (US); Christopher TOLLES, Cambridge, MA (US)

(21) Appl. No.: 17/133,460

(22) Filed: Dec. 23, 2020

Related U.S. Application Data
(60) Provisional application No. 62/954,128, filed on Dec. 27, 2019.

(51) Int. Cl. A61K 36/11; A61K 9/00; A61K 47/36; A61K 47/26; A61K 31/593; A61K 8/73; A61K 8/35; A61K 8/9794; A61P 39/06; A61Q 19/08; A61Q 19/00; A61K 8/02; A61K 8/9767; A61Q 19/02; A61K 8/31; A61K 8/42; A61K 31/047; A61K 31/4045; A61K 47/44; A61K 47/46; A61K 47/12; A61K 8/67; A61K 36/185; A23L 33/00; A23L 33/105; A23L 33/135; A23L 33/125; A23L 29/231; A23L 29/30; A23G 3/48; A23G 3/42; A23G 3/36

(52) U.S. Cl. CPC A61K 36/11; A23V 2002/00; A61K 47/36; A61K 47/26; A61K 31/593; A61K 8/732; A61K 8/355; A61K 8/9794; A61P 39/06; A61Q 19/08; A61Q 19/004; A61K 8/0216; A61K 8/9767; A61Q 19/02; A61Q 19/007; A61K 8/31; A61K 8/735; A61K 8/42; A61K 31/047; A61K 31/4045; A61K 47/44; A61K 47/46; A61K 47/12; A61K 8/678; A61K 36/185; A23L 33/40; A23L 33/105; A23L 33/135; A23L 33/125; A23L 29/231; A23L 29/30; A23G 3/48; A23G 3/42; A23G 3/366; A23G 3/368; A61K 2800/92; A61K 2800/48; A61K 9/0056

(57) ABSTRACT

A confection may include one or more skin protective ingredients, a thickening agent, and a sweetening agent. The one or more skin protective ingredients may include Polypodium leucotomos extract, astaxanthin, Pinus pinaster, lycopene, lutein, and/or zeaxanthin. In some embodiments, the confection may be formulated as a gummy, chewable, or candy, and may be taken orally on a regular basis (e.g., daily) to treat and/or prevent one or more skin conditions.

### CROSS-REFERENCE TO RELATED APPLICATIONS

This application claims priority to U.S. Patent Application Ser. No. 62/954,128 filed Dec. 27, 2019, which is incorporated herein in its entirety by this reference.

### TECHNICAL FIELD

This invention relates generally to the field of confections for health benefits.

### BACKGROUND

Environmental stressors can contribute to the development of a wide variety of dermatologic diseases. For example, light such as UVA light, UVB light, and/or infrared radiation can increase oxidative stress that may contribute to undesirable skin conditions. Other sources of light can also damage skin. For example, electronic devices (e.g., computers, laptops, tables, smartphones, etc.) emit high energy blue light that has been shown to cause skin aging. Additionally, air pollution contributes to oxidative stress via exposure to particulate matter and chemicals from environmental sources such as smog, automobile exhaust, cigarette smoke, and manufacturing processes. Many of these stressors are becoming more prevalent as a result of environmental changes such as climate change and increased urbanization.

Conventional approaches to reducing such environmental stressors include topical photoprotection, such as SPF-rated sunscreens, but such topical photoprotection has limitations. For example, topical photoprotection only protects against UVA and/or UVB light, leaving skin vulnerable to other stressors. Furthermore, adequate protection of skin against UVA and UVB light requires correct and diligent application of the topical photoprotection by a user, but this approach suffers from limited user compliance. It is estimated that very few people use sunscreen daily, and when they do use it, only apply 25-50% of what it needed to achieve adequate protection. Even at times of high sun exposure, such as a day at the beach, it is estimated that only 30% of people use sunscreen.

Accordingly, there is an urgent need for new and useful confections to improve skin protection and/or treatment strategies.

### SUMMARY

In some embodiments, a confection may include one or more skin protective ingredients having a weight concentration of less than 15% w/w, a thickening agent, and a sweetening agent. The one or more skin protective ingredients may be selected from the group consisting of: Polypodium leucotomos extract, astaxanthin, Pinus pinaster, lycopene, lutein, and zeaxanthin. In some embodiments, the confection may be formulated as a gummy, chewable, or candy.

For example, in some embodiments the one or more skin protective ingredients may include between about 60 mg and about 720 mg of Polypodium leucotomos extract, between about 1 mg and about 16 mg of astaxanthin, between about 10 mg and about 250 mg of extract from Pinus pinaster, between about 10 mg and about 50 mg of lycopene, between about 5 mg and about 30 mg lutein, and/or between about 0.5 mg and about 10 mg zeaxanthin.

In some embodiments, the thickening agent may include pectin, gelatin, sugar, alginates, agar, gum Arabic, alginic acid, sodium alginate, potassium alginate, calcium alginate, gellan gum, guar gum, carrageenan, locust bean gum, glucomannan, konjac, sodium carboxymethylcellulose, tapioca, tapioca syrup, tara gum, tamarind gum, starch, xanthan gum, maltodextrin, or a combination thereof. 8. In some embodiments, the thickening agent may have a weight concentration of between about 1% w/w to about 10% w/w.

In some embodiments, the sweetening agent may include cane sugar, maltodextrin, tapioca syrup, honey, sucrose, fructose, agave syrup, aspartame, alitame, attar, barley malt, beet sugar, birch syrup, blackstrap molasses, brazzein, brown rice syrup, brown sugar, cane juice, caramel, coconut palm sugar, high fructose corn syrup, curculin, date sugar or date honey, demerara sugar, dextrose, erythritol, fructose glucose syrup, fructose, galactose, sorbitol, glucose, glycerol, glycyrrhizin, grape syrup, isoglucose, inulin, inverted sugar, isomal, lactose, levulose, maltitol maltodextrin, maltose, mannitol, maple syrup, miraculin, molasses, monatin, monellin, monk fruit, neotame, oligofructose, palm sugar, pentadin, polydextrose, rapadura, rice syrup, saccharin, stevia, stevioside, sucralose, sugar beet syrup, tagatose, trehalose, xylitol, yacon syrup, or a combination thereof.

Furthermore, in some embodiments, the confection may further include one or more flavoring agents and/or additives. For example, the confection may include one or more additives such as a probiotic ingredient, a prebiotic ingredient, melatonin, a cannabinoid ingredient, an antioxidant, an anti-inflammatory ingredient, an ingredient to enhance skin barrier function, an ingredient to promote structural integrity of the extracellular matrix, or a combination thereof.

A method to treat or prevent a skin condition in a subject in need thereof, may include administering to the subject a confection including one or more skin protective ingredients having a weight concentration of less than 15% w/w, a thickening agent having a concentration of between about 1% w/w to about 15% w/w. and a sweetening agent. The one or more skin protective ingredients may be selected from the group consisting of: Polypodium leucotomos extract, astaxanthin, Pinus pinaster, lycopene, lutein, and zeaxanthin. In some embodiments, the confection may be formulated as a gummy, chewable, or candy. The confection may be orally administered to the subject daily.

For example, the skin condition may be contact dermatitis, alopecia areata, atopic dermatitis, seborrheic dermatitis, lichen planus, scleroderma, pemphigus, vitiligo, acne, rosacea, chronic venous ulcer, photosensitivity disorders, itch, cutaneous pain, disorders of impaired skin barrier function, psoriasis, disorders of oxidative stress in skin, radiation-induced skin damage, light-induced skin damage, oxidative skin damage, drug-induced photosensitivity, photosensitivity disorders, gut dysbiosis, photoaging, or aging.

### DETAILED DESCRIPTION

The following description sets forth numerous exemplary configurations, methods, parameters, and the like. It should be recognized, however, that such description is not intended as a limitation on the scope of the present disclosure but is instead provided as a description of exemplary embodiments.

Described herein are confections that that may, for example, reduce oxidative stress in skin when administered orally and exert a photoprotective effect on skin. The confections may enable high bioavailability and patient compliance. In some embodiments, a confection includes at least one skin protective ingredient (such as Polypodium leucotomos extract, astaxanthin, pycnogenol, lycopene, lutein or zeaxanthin), a thickening agent, a sweetener, and water or other liquid. In some embodiments, a confection may include one or more additive agents that augment the protective effects of the skin protective ingredients, such as other photoprotective agents, supplements, probiotics, prebiotics, cannabinoids, and/or vitamins to the confection formulation. The confections described herein have numerous advantages over conventional commercial formulations, which have been primarily limited to raw extract contained in a pill capsule. For example, confections are easier and more convenient for a typical user to consume, compared to pills that many people find challenging to swallow and/or fit into a consistent regimen. It has been estimated that up to 50% of the population has difficulty swallowing pills or capsules. Accordingly, suitable skin protective ingredient(s) in a confection form such as that described herein may lead to greater compliance for a more effective skin protective regimen.

Furthermore, the confections or other food formulations (e.g., gummy or chewable forms) described herein provide a greater bioavailability profile compared to pills. For example, when a skin protective ingredient is embedded in a gel matrix such as pectin, gelatin or agar, its dissolution is more distributed across the gastrointestinal tract relative to a raw ingredient encapsulated in a pill format. Furthermore, the act of chewing results in dissolution of the confection beginning in the mouth, where limited sublingual absorption can occur, which bypasses first-pass metabolism. Sublingual absorption is especially relevant for highly lipophilic skin protective ingredients, such as astaxanthin.

Methods of making a confection containing one or more skin protective ingredients are also described herein. Formulating skin protective ingredients in a confection form is not straightforward, as efficacy of such ingredients is unknown when the ingredients are simply added to a hot slurry to form a gummy or chewable, for example. Methods described herein enable the skin protective ingredients to withstand heat and retain their bioactive properties when being formulated into confection and other food formulations. Furthermore, the methods described herein can further improve bioavailability through facilitating sustained or targeted release of the select skin protective ingredient, which can be achieved by microencapsulation of the ingredient prior to adding to the confection, as described below. Microencapsulation of these ingredients augments their photoprotective and antioxidant effects in skin.

### Confection Composition

Provided herein are confections capable of reducing oxidative stress in skin and/or exerting a photoprotective effect. A confection may, for example, refer to a gummy, chewable, candy, comestible, food, or food ingredient for human or non-human (e.g., domesticated animals) consumption. The confection may possess any suitable flavor and/or aroma.

In some embodiments, a confection comprises one or more skin protective ingredients, a thickening agent, a sweetener agent, a flavoring agent, and/or water or other suitable liquid. In some embodiments, a confection may further include one or more additives (e.g., stabilizers, buffers, compounds to augment skin protection, supplements, vitamins, etc.). Examples and further details of these ingredients in a confection composition are described further below.

### Skin Protective Ingredients

In some embodiments, the skin protective ingredient(s) in a confection may include, for example, extract from the family Polypodium (e.g., Polypodium leucotomos extracts), astaxanthin, Pinus pinaster (otherwise known as P. maritima, which includes the extract pycnogenol), lycopene, lutein, or zeaxanthin. These specific ingredients retain their skin protective properties after being added to a hot slurry and enduring the gelation process for a confection.

For addition to confection formulations, the skin protective ingredient(s) may be in the form of powders, solids, liquids, emulsions, or tinctures. In some embodiments of a powdered format, it may pass through a mesh size of 5, 10, 20, 30, 40, 50, 60, 70, 80, 90, 100, 150 or 200. In some embodiments of a powdered format, it may pass through a mesh size of 60 or smaller. These ingredients may also be encapsulated, microencapsulated and/or nanoencapsulated in order to improve bioavailability, clinical efficacy, taste profile or feasibility for mixture into the confection. Examples of encapsulation methods include: encapsulating a core of one or more of the selected skin protective ingredients in a single membrane, encapsulating a core of one or more selected skin protective ingredient(s) in a multilayer or multiple shell capsule, and dispersing of one or more select skin protective ingredients in a polymer matrix. Examples of suitable encapsulation agents include, but are not limited to, amylose, pectin, alginates, agar, carrageenan, caseinate, whey, cellulose, cellulose derivatives cyclodextrin, maltodextrin, inulin, albumin, lactose, polyvinyl alcohol, polyethylene glycol, lecithin, chistosan(s), gelatin, polyesters (PLGA, PLLA, PCL, PPE), polyorthoesters (POE I-IV), polyanhydrides, polyphosphazines, gums, starches, sugar derivatives, shellac, and zein.

In some embodiments, the skin protective ingredient(s) may not exceed 10% w/w, 15% w/w, or 20% w/w of total target confection weight. In some embodiments, the amount of selected skin protective ingredient is less than 10% w/w of the target confection weight. For example, if target confection weight is 2.5 g then no more than a total of 250 mg of skin protective ingredient(s) may be added to the confection.

In some embodiments, the skin protective ingredient(s) includes Polypodium leucotomos extract (PLE). Polypodium leucotomos extract is the extract of a fern endemic to the Americas. Polypodium leucotomos has been extensively studied for its photoprotective properties when applied to the skin topically or ingested orally in pill format. However, PLE has not been formulated into one of these user-friendly formats of a confection. PLE is best characterized for its photo protective properties; however, there are other ferns in the genus Polypodium that are thought to have similar photoprotective properties to PLE that may also be incorporated into a confection. Ferns similar to Polypodium leucotomos, are Polypodium aureum, Polypodium crassifolium, Polypodium decumanum, Polypodium lanceolatum, Polypodium leucotomos, Polypodium percussum, Polypodium triseriale, or Polypodium vulgare may also be used to achieve therapeutic benefits. Extracts of PLE or related ferns for use in a confection formulation may be obtained from the aerial parts of the plant (such as the fronds) or the rhizome. In some embodiments each confection may contain between about 10 mg to about 3000 mg, between about 15 mg to about 2800 mg, between about 30 mg to about 1400 mg, between about 60 mg to about 700 mg, or between about 120 mg to about 350 mg of Polypodium leucotomos extract. Exemplary embodiments may contain a minimum of about 60 mg of Polypodium leucotomos extract, to a maximum of about 720 mg, per confection. In some embodiments, each resultant confection will contain about 240 mg of Polypodium leucotomos extract. In some embodiments weight ranges of a single confection range from between about 125 mg to about 40 g, between about 250 mg to about 20 g, between about 500 mg to about 10 g, between about 1 g to about 5 g, or between about 2 g to about 2.5 g. In some embodiments weight ranges of a single confection range from between about 500 mg to about 10 g.

The potent antioxidants contained in Polypodium leucotomos extract have been shown to reduce UV-induced cell damage, reduce oxidative stress, reduce DNA damage, block UV radiation-induced immune suppression, and inhibit the release of UV-induced levels of cyclooxygenase-2 and inflammatory cytokines. Polypodium leucotomos extract is well established to photoprotect skin from ultraviolet radiation when ingested orally, however embodiments have centered on pharmaceutical carriers such as pills, which are associated with poor patient compliance. The most established use of PLE is for protecting the skin against the harmful effects of UV radiation, which includes UVB and UVA radiation. PLE assists in the prevention and accelerates repair of DNA damage associated with sun exposure, as well as mitigates the substantial oxidative damage associated with sun exposure. Hence, the PLE-containing confection may be used for the purpose of providing oral photoprotection. Given the mechanism of action, of enhancing DNA repair and mitigating oxidative damage, PLE-containing confections may also be used for the prevention and treatment of photoaging. Additionally, given the mechanism of action of enhancing DNA repair, the PLE-containing confection may be used for the chemoprevention of non-melanoma skin cancers, such as basal cell carcinoma and squamous cell carcinoma. The PLE-containing confection may also be used to prevent and treat skin aging associated with high energy visible light and environmental pollution. The PLE confection may also be used to treat photosensitive disorders, such as dermatomyositis, lupus erythematosus, pemphigus, polymorphic light eruption, melasma, pseudoporphyria, and solar urticaria. It may also be used to treat drug-induced photosensitivities, caused by drugs such as, but not limited to, doxycycline, tertracycline, ciprofloxacin, levofloxacin, sulfonamides, ketoprofen, naproxen, celecoxib, furosemide, bumetanide, hydrochlorothiazide, glipizide, glyburide, atorvastatin, pravastatin, simvastatin, chlorpromazine, thioridazine, chlorprothixene, thiothixene, terbinafine, itraconazole, voriconazole, griseofulvin, 5-flourouracil, paclitaxel, amiodarone, diltiazem, quinidine, dapsone and oral contraceptives.

In some embodiments, the skin protective ingredient(s) includes astaxanthin. Astaxanthin is a carotenoid which is most commonly derived from Haematococcus pluvialis, a type of freshwater algae. Other natural sources of astaxanthin include Chlorella zofingiensis, Chlorococcum, and Phaffia rhodozyma. Astaxanthin is a potent antioxidant from the carotenoid family. It is known for its bright red color and is the compound responsible for giving salmon and shrimp their pinkish hue. Astaxanthin has been shown to be a more powerful antioxidant than vitamins A, C, E, B-carotene, resveratrol, green tea, or coenzyme Q10. Hence, astaxanthin is capable of reducing oxidative stress in cells and has potent anti-inflammatory and immunomodulatory effects. At the cellular level, astaxanthin works to boost skin's natural defense and repair mechanisms to combat the toll that oxidative stress takes on skin health and appearance. Astaxanthin has been shown to have an excellent safety and tolerability profile and has been demonstrated to be bioavailable. Daily oral administration of astaxanthin has been shown to have the following anti-aging benefits based on study endpoints: a decrease in systemic markers of oxidative stress (malondialdehyde (MDA)), decrease in appearance of wrinkles, decrease in age spot size, increased skin hydration as evidenced by decreases in transepidermal water loss, protects skin against deterioration from UV-light as evidenced by increases in minimal erythema dose (MED), increased skin elasticity, increased production of new collagen as evidenced by increases in procollagen type I and decreases in enzymes associated with collagen and elastin degradation such as MMP-1 and MMP-12. Delivery mechanisms for astaxanthin have been largely limited to pills and powders, which are associated with poor compliance. Other applications for which astaxanthin has been demonstrated to have beneficial health effects in humans include enhanced cell-mediated and humoral immunity decreased gastric inflammation, and preliminary research indicates astaxanthin may have antidiabetic, anticarcinogenic, anti-lipid peroxidation activity, and place a role in promoting healthy cardiometabolic function. In some embodiments, each resultant confection may contain between about 0.25 mg to about 64 mg, between about 0.5 mg to about 32 mg, between about 1 mg to about 16 mg, or between about 2 mg to about 8 mg of astaxanthin. Exemplary embodiments will contain a minimum of about 1 mg of astaxanthin to a maximum of about 16 mg, per confection. In some embodiments, each resultant confection will contain about 4 mg of astaxanthin.

In some embodiments, the skin protective ingredient(s) includes an extract from Pinus pinaster, also known as Pinus maritima, which includes pycnogenol and flavagenol. P. pinaster extract is rich in potent antioxidants such as bioflavonoids and oligometric proanthocyanidins (OPCs) which reduce oxidative stress and are thought to be responsible for the therapeutic effects of the extract. In some embodiments the skin protective ingredient(s) is an extract derived from the bark of P. pinaster, such as pycnogenol or flavagenol. Oral administration of extracts of P. pinaster has been demonstrated to decrease the visible signs of photoaging in skin, photoprotect skin, reduce hyperpigmentation, even skin tone, improve skin elasticity, improve skin hydration, promote hyaluronic acid synthesis, and promote collagen synthesis. P. pinaster extract has also demonstrated to be of therapeutic value in pigmentary disorders such as melasma. Other applications for which extracts of P. maritima have been demonstrated to have beneficial health effects in humans include therapeutic value in the treatment of osteoarthritis, atherosclerosis, neuroprotection, as well as potent immunomodulatory and anti-inflammatory effects that have been supported by meta-analyses. In some embodiments each confection may contain between about 2.5 mg to about 1 g, between about 5 mg to about 500 mg, between about 10 mg to about 250 mg, between about 20 mg to about 125 mg, or between about 40 mg to about 62.5 mg of P. pinaster extract. Exemplary embodiments will contain a minimum of 10 mg of P. pinaster extract to a maximum of 250 mg, per confection. In some embodiments, each resultant confection will contain about 40 mg of P. pinaster extract. In some embodiments each confection may contain between about 2.5 mg to about 1 g, between about 5 mg to about 500 mg, between about 10 mg to about 250 mg, between about 20 mg to about 125 mg, or between about 40 mg to about 62.5 mg of the P. pinaster extract pycnogenol. Exemplary embodiments contain a minimum of 10 mg of the P. pinaster extract pycnogenol to a maximum of 250 mg, per confection. In some embodiments, each resultant confection contains about 40 mg of the P. pinaster extract pycnogenol.

In some embodiments, the skin protective ingredient(s) includes lycopene. Lycopene is a carotenoid known to have potent antioxidant, ant-inflammatory and immunomodulatory effects. Oral administration of lycopene has been demonstrated to decrease the visible signs of photoaging in skin, photoprotect skin, even skin texture, improve skin elasticity, improve skin hydration, and promote collagen synthesis. In some embodiments each confection may contain between about 1 mg to about 100 mg, contain between about 2 mg to about 50 mg, contain between about 5 mg to about 25 mg, or contain between about 10 mg to about 12 mg lycopene. Exemplary embodiments will contain a minimum of about 5 mg of lycopene to a maximum of about 25 mg, per confection. In some embodiments, each resultant confection will contain about 10 mg of lycopene.

In some embodiments, the skin protective ingredient(s) includes lutein, zeaxanthin, or combinations thereof. Of note, lutein and zeaxanthin are isomeric compounds that absorb blue light, making them of interest in reducing oxidative stress in skin secondary to high energy blue light. The protective effects of lutein and zeaxanthin on decreasing oxidative stress in eye tissue associated with blue light are well established, and emerging data suggests this benefit can extend to skin. Lutein and zeaxanthin are carotenoids known to have potent antioxidant, anti-inflammatory and immunomodulatory effects. Oral administration of lutein and zeaxanthin have been shown to decrease the visible signs of photoaging in skin, photoprotect skin, and protect skin against blue light associated oxidative stress. In some embodiments each confection may contain between about 1 mg to about 80 mg, between about 2 mg to about 40 mg, or between about 4 mg to about 20 mg lutein. In some embodiments each confection may contain between about 0.1 mg to about 40 mg, between about 0.2 mg to about 20 mg, between about 0.5 mg to about 10 mg, or between about 1 mg to about 5 mg zeaxanthin. In some embodiments, each resultant confection will contain about 10 mg of lutein and 2 mg of zeaxanthin. Exemplary embodiments contain a minimum of about 5 mg of lutein to a maximum of about 20 mg, per confection, and a minimum of about 0.5 mg to a maximum of 10 mg of zeaxanthin per confection.

### Thickening Agents

In some embodiments the confection comprises one or more thickening agents or gelling agents. Exemplary thickening agents may include pectin, gelatin, sugar, or combinations thereof. Other thickening agents that may additionally or alternatively be used include: alginates, agar, gum Arabic, alginic acid, sodium alginate, potassium alginate, calcium alginate, gellan gum, guar gum, carrageenan, locust bean gum, glucomannan, konjac, sodium carboxymethylcellulose, tapioca, tapioca syrup, tara gum, tamarind gum, starch, xanthan gum, maltodextrin, and combinations thereof. If pectin is selected, fruit pectin may be advantageous due to its production of an elastic-viscous texture.

### Sweetening Agents

In some embodiments the confection comprises one or more sweetening agents. Examples of sweetening agents may include cane sugar, maltodextrin, tapioca syrup, honey, and combinations thereof. Other potential embodiments may use: sucrose, fructose, agave syrup (e.g., agave nectar), aspartame, alitame, attar, barley malt (e.g., barley malt syrup), beet sugar, birch syrup, blackstrap molasses, brazzein, brown rice syrup, brown sugar, cane juice (e.g., evaporated cane juice), caramel, coconut palm sugar, high fructose corn syrup, curculin, date sugar or date honey, demerara sugar, dextrose, erythritol, fructose glucose syrup, fructose, galactose, sorbitol, glucose, glycerol, glycyrrhizin, grape syrup, isoglucose, inulin, inverted sugar, isomal, lactose, levulose, maltitol maltodextrin, maltose, mannitol, maple syrup, miraculin, molasses, monatin, monellin, monk fruit, neotame, oligofructose, palm sugar (e.g., palm syrup), pentadin, polydextrose, rapadura, rice syrup, saccharin, stevia, stevioside, sucralose, sugar beet syrup, tagatose, trehalose, xylitol, yacon syrup, or combinations thereof.

### Flavoring Agents

In some embodiments the confection comprises one or more flavoring agents. Exemplary flavoring agents include natural oils, such as organic orange oil, or other naturally derived flavors. The confection may possess any taste or flavor. In some embodiments the confection may have a flavor such as sweet, sour, tangy, salty, spicy, and bitter. The flavor may include one or more suitable flavors such as citrus, berry flavors (e.g., blueberry, strawberry, acai, raspberry, etc.), other fruit flavors (e.g., cherry, apple, peach, grape, etc.), menthol, peppermint, chocolate, spice flavors (e.g., cinnamon, nutmeg, etc.), oil flavors (lemon oil, ginger oil, etc.). In some embodiments, the confection may have no taste or flavor. In some embodiments the confection may have a blackberry hibiscus flavor. In some embodiments the confection may have an orange bergamot flavor.

### Additives

In some embodiments the confection comprises one or more additives. Examples of additives may include stabilizers, preservatives buffers. Additionally or alternatively, a confection may include additives including additional agents such as compounds or other substances to augment skin protection, supplements and vitamins, as described in further detail below.

In some embodiments the total predetermined amount of additive, or combinations of additives, may be less than 2% w/w, 4% w/w, 6% w/w, 8% w/w, 10% w/w, 12% w/w, 14% w/w, 16% w/w, 18% w/w, or 20% w/w of target confection weight. In some embodiments, the total predetermined amount of skin protective additive, or combinations of additives, may be less than 15% w/w of target confection weight. In one example, if target confection weight is 2.5 g then the maximum allowable amount of additive for each individual confection is 375 mg

In some embodiments, the confection includes one or more stabilizers. For example, in some embodiments the confection comprises sodium citrate as a stabilizing agent in order to obtain an elastic-viscous texture. Other exemplary stabilizers include antioxidants to maintain skin protective ingredient stability in the confection matrix, such as tocopherols, ascorbic acid, sodium ascorbate, butylated hydroxytoluene, butylated hydroxyanisole, gallic acid, sodium gallate, or sulfites. In some embodiments the antioxidant is mixed tocopherols.

Other inactive ingredients that may be added as a preservative, stabilizer, flavoring, or coloring agent. These may include but are not limited to: citric acid, lactic acid, tartaric acid, calcium salts, beeswax, and carnauba wax. Citric acid, lactic acid, phosphoric acid and tartaric acid may be used as retarders and stabilize pH. Other buffering agents may include, potassium citrate, seignette salt, sodium lactate and sodium citrate. The confection may also be coated with a natural wax or sugar to prevent agglomeration and sticking.

### Additional Agents

Additionally or alternatively, a confection may include additives including additional agents such as compounds or other substances to augment skin protection, supplements and vitamins.

For example, in some embodiments, the confection comprises melatonin. Blue light is known to disrupt endogenous melatonin production, thereby contributing to insomnia, and consequently increased systemic oxidative stress. Accordingly, the addition of melatonin may be help counteract the effects of blue light on skin. In some embodiments a confection may contain between about 0.1 mg to about 10 mg, between about 0.1 mg to about 7 mg, between about 0.1 mg to about 5 mg, or between about 0.5 mg to about 3 mg melatonin.

In one illustrative example of a confection optimized for mitigating oxidative stress due to blue light, the confection may include water, a sweetening agent, a thickening agent, a minimum of between about 5 mg to a maximum of about 20 mg of lutein, per confection, and a minimum of between about 0.5 mg to a maximum of about 5 mg of zeaxanthin per confection, and a minimum of between about 0.1 mg to a maximum of about 5 mg of melatonin per confection. In some embodiments to mitigate the deleterious health effects of blue light on skin, the confection contains water, a sweetening agent, a thickening agent, about 10 mg of lutein, about 2 mg of zeaxanthin and about 1 mg of melatonin. For these embodiments, any suitable sweetening agents and thickening agents such as those described above may be included in the confection.

In some embodiments the confection comprises one or more antioxidant-rich compounds. In some embodiments, antioxidant-rich compounds capable of reducing oxidative stress in skin and capable of exerting a photoprotective effect on skin may be added to the confection mixture to enhance the skin protective effect, these may include one or more of the following: goji berry (Lycium barbarum), pomegranate (Punica granatum L), garlic, canthaxanthin, B-carotene, lycopene, ascorbic acid (vitamin C), niacinamide (vitamin B3), tocopherols (vitamin E), silibinin, silymarin, epigallocatechin-3-gallate (green tea polyphenol), curcumin, equol, quercetin, apigenin, genistein, rosmarinic acid, uncaria tomentosa extract, reservatrol, perillyl alcohol, limonene, caffeine, and selenium.

In some embodiments the confection comprises one or more anti-inflammatory ingredients. Anti-inflammatory ingredients that may be suitable for inclusion include: Uncaria tomentosa, Uncaria guianensis, Capsicum annum, Capsicum Frutescens, Polygonum cuspidatum, Boswellia serrata, Curcuma longa, Camellia sinensis, Salix Alba, or Harpagophytum Procumbens.

In some embodiments the confection comprises one or more vitamins. In some embodiments, vitamins may be added to the confection, such as Vitamin D compounds, such as ergocalciferol or cholecalciferol. Vitamin D is of particular interest, as fear of Vitamin D deficiency is cited as a common reason that people do not utilize photoprotection strategies and it is known to have improved bioavailability when administered in confection format. Furthermore, additional embodiments include the addition of other additives to the user-friendly format, including vitamin D (cholecalciferol) and its analogs.

In some embodiments the confection comprises one or more ingredients to improve skin barrier function and promote structural integrity of the extracellular matrix. In some embodiments, a confection could contain an additional ingredient or combination of ingredients shown to improve skin barrier function and promote structural integrity of the extracellular matrix.

An illustrative example of a confection optimized for mitigating oxidative damage to the extracellular matrix includes water, a sweetening agent, a thickening agent, a skin protection agent selected from one or more of the following Polypodium leucotomos extract, astaxanthin, extract from Pinus pinaster also known as Pinus maritima, lycopene, lutein or zeaxanthin and one or more additional ingredients selected from the following: low molecular weight hyaluronic acid, phytoceramides, alpha-linolenic acid (ALA), eicosapentaenoic acid (EPA), docosahexaenoic acid (DHA), or marine collagen peptides. Oxidative stress is also known to damage the structural proteins of the extracellular matrix (ECM) which form the foundational structure of skin. The extracellular matrix is composed of collagens (Type I, III, IV, V, VI, VII), elastic fibers (elastin, microfibrillar components and fibrillin), and basement membrane associated macromolecules (proteoglycans, glycosaminoglycans such as hyaluronic acid, laminin, fibronectin), and ceramides. In fact, the extracellular matrix is known to be particularly sensitive to oxidative stress to its lower concencentration of endogenous protective mechanisms such as antioxidants and repair enzymes. In this embodiment the skin protective ingredient (one or more selected from: Polypodium leucotomos extract, astaxanthin, extract from Pinus pinaster also known as Pinus maritima, lycopene, lutein or zeaxanthin) would decrease oxidative stress at the site of the extracellular matrix via an antioxidant mechanism, and the additional ECM-promoting ingredient to be selected from low molecular weight hyaluronic acid, phytoceramides, alpha-linolenic acid (ALA), eicosapentaenoic acid (EPA), docosahexaenoic acid (DHA), or marine collagen peptides will increase the availability of structural components to repair and replace ECM components depleted by oxidative stress and achieve a synergistic effect. In some embodiments, the ECM-promoting ingredients will be a combination of very low molecular weight hyaluronic acid (<10,000 Daltons) and a phytoceramide.

In some embodiments the confection comprises one or more probiotic agents. In some embodiments, a confection may contain a probiotic ingredient. A probiotic ingredient augments the anti-inflammatory and antioxidant effects of the skin protective ingredient(s) by decreasing gut dysbiosis and gut dysbiosis-associated inflammation. It is known that dysbiosis can have potent inflammatory and prooxidant effects. The combination of a skin protective antioxidant in a confection matrix and an agent to promote a healthy gut microbiome would decrease oxidative stress associated with dysbiosis and improve skin health and appearance.

An illustrative example of a confection optimized to reduce oxidative stress in skin via decreasing oxidative stress in the gut and promoting a healthy gut microbiome, would include water, a sweetening agent, a thickening agent, a skin protection agent selected from one or more of the following Polypodium leucotomos extract, astaxanthin, extract from Pinus pinaster also known as Pinus maritima, lycopene, lutein or zeaxanthin and at least one probiotic ingredient to be selected from the following: Lactobacillus (Lact. acidophilus, Lact. brevis, Lact. casei, Lact. curvatus, Lact. fermentum, Lact. gasseri, Lact. Johnsonii, Lact. reuteri, Lact. rhamnosus, Lact. salivarius), Bifidobacterium (Bif. adolescentis, Bif. animalis, Bif. breve, Bif. infantis, Bif. longum, Bif. thermophilum), Enterococcus (Ent. faecalis, Ent. faecium), Streptococcus (Strep. thermophilus), or Lactococcus (L. lactis subspecies cremoris, L. lactis subspecies lactis). In some embodiments the probiotic ingredient is Lact. Johnsonii and/or Lact. rhamnosus. It is anticipated that probiotics administered to the gut will decrease oxidative stress, and associated diseases, in skin.

In some embodiments the confection comprises one or more prebiotic ingredients. In some embodiments, a confection may contain a prebiotic ingredient. A prebiotic ingredient augments the antinflammatory and antioxidant effects of the skin protective ingredient(s) by decreasing gut dysbiosis and gut dysbiosis-associated inflammation. It is known that dysbiosis can have potent inflammatory and prooxidant effects. The combination of a skin protective antioxidant in a confection matrix and an agent to promote a healthy gut microbiome would decrease oxidative stress associated with dysbiosis and improve skin health and appearance.

An illustrative example of a confection optimized to reduce oxidative stress in skin via decreasing oxidative stress in the gut and promoting a healthy gut microbiome, includes water, a sweetening agent, a thickening agent, a skin protection agent selected from one or more of the following Polypodium leucotomos extract, astaxanthin, extract from Pinus pinaster also known as Pinus maritima, lycopene, lutein or zeaxanthin and at least one prebiotic ingredient to be selected from the following: oligosaccharide carbohydrates such as fructans (e.g., inulin or oligofructose), galacto-oligosaccharides, starch and glucose-derived oligosaccharides (e.g., resistant starch or polydextrose) or other oligosaccharides.

In some embodiments the confection comprises one or more cannabinoid ingredients. In some embodiments, a confection may contain a cannabinoid ingredient. Oral administration of cannabinoids has been shown to play a role in immunomodulation of skin to decrease oxidative stress and promote skin health, as well as to improve the overall cosmetic appearance of skin decrease inflammation associated with oxidative stress in skin, and promote appropriate immune response to skin stressors. In fact, dysregulation of the cutaneous cannabinoid system signaling has been associated with inflammation, oxidative stress and is also associated with the development of dermatologic diseases such as atopic dermatitis, psoriasis, acne, pigmentary disorders, scleroderma, disorders of barrier function, itch and cutaneous pain. Hence, exogenous phytocannabinoids can play a key role in combination with skin protective antioxidants in reducing oxidative stress in skin and promoting skin health.

An illustrative example of a confection optimized to reduce oxidative stress in skin via promotion of healthy cutaneous cannabinoid signalling and immunomodulation, includes water, a sweetening agent, a thickening agent, a skin protection agent selected from one or more of the following Polypodium leucotomos extract, astaxanthin, extract from Pinus pinaster also known as Pinus maritima, lycopene, lutein or zeaxanthin and at least one cannabinoid ingredient to be selected from the following: exemplary phytocannabinoids include: tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV, cannabidivarin (CBDV), cannabichromevarin (CBCV), cannabigerovarin (CBGV), cannabigerol monomethyl ether (CBGM), cannabielsoin (CBE), and cannabicitran (CBT). In some embodiments the cannabinoids would be one or more of the following: cannabidiol (CBD), delta-9-tetrahydrocannabinol (Δ9-THC) or cannabigerol (CBG). In some embodiments, the confection may include an amount of one or more cannabinoid ingredients that is between about 10 mg and about 40 mg, between about 15 mg and about 35 mg, between about 10 mg and about 35 mg, between about 10 mg and about 25 mg, between about 10 mg and about 2 mg, between about 20 mg and about 40 mg, or between about 25 mg and about 40 mg or about 30 mg and about 40 mg.

In some embodiments the confection comprises nutraceuticals (e,g., ginseng, ginkgo biloba, etc.), minerals (e.g., calcium, magnesium, etc.) energizing agents (e,g., caffeine, taurine, etc.), chemesthesis ingredients (e.g., capsaicin, peppermint, etc.), plant and food oils (mustard oil, wintergreen oil, etc.), amino acids (e.g., branched amino acids, essential amino acids, etc.), soothing agents (e.g., jasmine, lavender, etc.), emulsifiers (e.g., polysorbates, phospholipids), pH modifiers (e.g., sodium bicarbonate, etc.), or combinations thereof.

### Method of Making a Confection

Provided herein are methods to make a confection. In some embodiments the method comprises mixing a predetermined amount of water, thickening agent(s), sweetener(s), flavoring agents, or combinations thereof, and heating the mixture to a boiling, slurry-like consistency.

Predetermined amounts of skin-protective ingredients may then be added to the slurry post-boiling. Addition of the skin protective ingredient(s) following boiling ensures the integrity of the active antioxidant compounds contained in the extract. Addition of the skin protective ingredient(s) prior to boiling dramatically decreases the efficacy of the resulting confection due to potential degradation of active organic compounds.

Predetermined amounts of additional additives such as: ingredients that augment skin protection effects, stabilizers, additional photoprotective agents, cannabinoids, flavoring agents, and vitamins, or combinations thereof may also be added to the slurry post-boiling. Any of the above-described additives may be added to the slurry.

In some embodiments the total predetermined amount of skin protective ingredient, or combinations of skin protective ingredients, may be less than 5% w/w, 10% w/w, 15% w/w, or 20% w/w of target confection weight. In some embodiments the total predetermined amount of additive, or combinations of additives, may be less than 2% w/w, 4% w/w, 6% w/w, 8% w/w, 10% w/w, 12% w/w, 14% w/w, 16% w/w, 18% w/w, or 20% w/w of target weight. In some embodiments the total predetermined amount of additive, or combinations of additives, may be less than 10% w/w of target weight. In an exemplary embodiment, if a target confection weight is 2.5 g then the maximum allowable amount of additive for each individual confection is 375 mg. In another exemplary embodiment, if a target confection weight is 3 g then the maximum allowable amount of additive for each individual confection is 300 mg.

The mixture is then cooled and molded to form the confection. The confection may be molded into any desired format, such as gummies, chewable, candies, chewing gum, etc. The confection may be molded in any desired shape, such as a square, rectangle, circle, triangle, star, etc. A person of skill in the art would recognize the various format and shapes for molding the confection.

The resulting solution will be mixed, cooled, and the predetermined amount of skin protective ingredient(s) will be added to the confection slurry post-boiling, but prior to cooling to room temperature.

### Applications

Provided herein are methods to treat a dermatologic condition in a subject or patient in need thereof, comprising administering the confections disclosed herein. Oxidative stress in known to exacerbate or be involved in the pathogenesis of many dermatologic diseases. Exemplary skin diseases associated with increases in oxidative stress that may benefit from administration of one of the confections described herein, include, contact dermatitis, alopecia areata, atopic dermatitis, seborrheic dermatitis, lichen planus, scleroderma, pemphigus, vitiligo, acne, rosacea, chronic venous ulcer, photosensitivity disorders, itch, cutaneous pain, disorders of impaired barrier function, and psoriasis.

Further, administration of the confections described herein can serve to decrease oxidative stress in skin caused by environmental factors and/or treat or mitigate dermatologic diseases associated with increased oxidative stress burden. They may also be administered for purposes such as: decreasing oxidative stress in skin, photoprotecting skin, decreasing radiation-induced skin damage, decreasing oxidative skin damage due to the environment, improving health, treating drug-induced photosensitivity, treating photosensitivity disorders, treating a dermatologic disorder, treating gut dysbiosis, decreasing the appearance of photoaging, and improving overall skin appearance. In some embodiments the confections described herein may be administered to a subject or patient to improve the cosmetic appearance of skin.

The confections may be administered to the subject or patient orally in any appropriate dosing regimen. In some embodiments the confection may be administered daily. In some embodiments the confection may be administered one or more times per day. In some embodiments the confection may be administered weekly. In some embodiments the confection may be administered bi-weekly. In some embodiments the confection may be administered every other week. In some embodiments the confection may be administered at any suitable time of the day, such as in the morning or at night, before or after breakfast, etc.

The confections may be administered as a unit dose. The term “unit dose” or “unit dosage” means a dosage form containing an amount of the confection described herein in one single dose. The unit dose may be, for example, in the form of one or more confections. The unit dose for a daily administration herein may be divided across multiple confections; for example, two confections (each capsule including about half of the unit dose) may be designed to be orally administered on the same day.

Unless otherwise defined, this application uses standard definitions. A subject or patient may refer to a human being.

### EXAMPLES

The following examples are merely illustrative and are not meant to limit any aspects of the present disclosure in any way.

### Example 1

A gummy containing 240 mg of Polypodium leucotomos extract, 20 mcg of cholecalciferol, where the thickener is fruit pectin, where the sweeteners are tapioca and cane sugar, is taken daily by a subject or patient to provide photoprotection and mitigate the effects of oxidative stress on skin.

### Example 2

A gummy containing 4 mg astaxanthin, with pectin as a thickening agent, where the sweeteners are cane juice and rice syrup, is taken daily by subjects or patients to mitigate the effects of skin aging caused by UV radiation, high energy visible light, environmental pollution, or any combination of thereof.

### Example 3

A gummy containing 40 mg of pycnogenol, with pectin as a thickening agent, where the sweeteners are cane juice and rice syrup, is taken daily by subjects or patients to visibly even skin tone.

### Example 4

A gummy containing at least 10 mg of lycopene, 60 mg of low molecular weight hyaluronic acid (<10,000 daltons), and 30 mg of phytoceramides to decrease oxidative stress on skin extracellular matrix, improve skin barrier function, and/or improve skin hydration.

### Example 5

A gummy with pectin as a thickening agent, containing 10 mg of lutein, 2 mg of zeaxanthin, and 1 mg of melatonin is administered nightly to mitigate oxidative stress caused by blue light and/or enhance sleep.

### Example 6

A gummy containing total carbohydrate 2 g (1% Daily Value), Total Sugars 1 g (including 1 g added sugars, 2% Daily Value), Vitamin D3 (as cholecalciferol), 20 mcg (100% DV), and Polypodium leucotomos extract (rhizome) 240 mg. Other ingredients include organic brown rice syrup, organic evaporated cane juice, water, natural orange and bergamot flavor with other natural flavors, organic orange juice concentrate, fruit pectin, citric acid, sunflower oil, carnauba leaf wax (to prevent sticking). The gummy is taken daily per day by subjects or patients in the morning to provide photoprotection and mitigate the effects of oxidative stress on skin. The gummy has a orange bergamot flavor. Percent Daily Values are based on a 2,000 calorie diet.

### Example 7

A gummy containing total carbohydrate 2 g (1% Daily Value), total sugars 1 g (including 1 g added Sugars, 2% Daily Value), and natural Astaxanthin (from H. pluvialis algae) 4 mg. Other ingredients include organic brown rice syrup, organic evaporated cane juice, water, fruit pectin, organic blackberry and hibiscus flavor with other natural flavors, organic orange juice concentrate, citric acid, vegetable oil (from coconut and canola), carnauba leaf wax (to prevent sticking), D-alpha tocopherol, and mixed tocopherols. The gummy is taken daily per day by subjects or patients to mitigate the effects of skin aging caused by UV radiation, high energy visible light, environmental pollution, or any combination of thereof. The gummy has a Blackberry Hibiscus flavor. % Daily Values are based on a 2,000 calorie diet.

What is claimed is:

1. A confection comprising:
one or more skin protective ingredients having a weight concentration of less than 15% w/w,
wherein the one or more skin protective ingredients are selected from the group consisting of: Polypodium leucotomos extract, astaxanthin, Pinus pinaster, lycopene, lutein, and zeaxanthin;
a thickening agent; and
a sweetening agent.

2. The confection of claim 1, wherein the one or more skin protective ingredients comprises between about 60 mg and about 720 mg of Polypodium leucotomos extract.

3. The confection of claim 1, wherein the one or more skin protective ingredients comprises between about 1 mg and about 16 mg of astaxanthin.

4. The confection of claim 1, wherein the one or more skin protective ingredients comprises between about 10 mg and about 250 mg of extract from Pinus pinaster.

5. The confection of claim 1, wherein the one or more skin protective ingredients comprises between about 10 mg and about 50 mg of lycopene.

6. The confection of claim 1, wherein the one or more skin protective ingredients comprises between about 5 mg and about 30 mg lutein.

7. The confection of claim 1, wherein the one or more skin protective ingredients comprises between about 0.5 mg and about 10 mg zeaxanthin.

8. The confection of claim 1, wherein the thickening agent comprises pectin, gelatin, sugar, alginates, agar, gum Arabic, alginic acid, sodium alginate, potassium alginate, calcium alginate, gellan gum, guar gum, carrageenan, locust bean gum, glucomannan, konjac, sodium carboxymethylcellulose, tapioca, tapioca syrup, tara gum, tamarind gum, starch, xanthan gum, maltodextrin, or a combination thereof.

9. The confection of claim 1, wherein the sweetening agent comprises cane sugar, maltodextrin, tapioca syrup, honey, sucrose, fructose, agave syrup, aspartame, alitame, attar, barley malt, beet sugar, birch syrup, blackstrap molasses, brazzein, brown rice syrup, brown sugar, cane juice, caramel, coconut palm sugar, high fructose corn syrup, curculin, date sugar or date honey, demerara sugar, dextrose, erythritol, fructose glucose syrup, fructose, galactose, sorbitol, glucose, glycerol, glycyrrhizin, grape syrup, isoglucose, inulin, inverted sugar, isomal, lactose, levulose, maltitol maltodextrin, maltose, mannitol, maple syrup, miraculin, molasses, monatin, monellin, monk fruit, neotame, oligofructose, palm sugar, pentadin, polydextrose, rapadura, rice syrup, saccharin, stevia, stevioside, sucralose, sugar beet syrup, tagatose, trehalose, xylitol, yacon syrup, or a combinations thereof.

10. The confection of claim 1, further comprising a flavoring agent.

11. The confection of claim 10, wherein the one or more additives.

12. The confection of claim 10, wherein the one or more additives comprises a probiotic ingredient.

13. The confection of claim 10, wherein the one or more additives comprises a prebiotic ingredient.

14. The confection of claim 10, wherein the one or more additives comprises melatonin.

15. The confection of claim 10, wherein the one or more additives comprises a cannabinoid ingredient.

16. The confection of claim 11, wherein the one or more additives comprises an antioxidant, an anti-inflammatory ingredient, an ingredient to enhance skin barrier function, an ingredient to promote structural integrity of the extracellular matrix, or a combination thereof.

17. The confection of claim 1, wherein the confection is formulated as a gummy, chewable, or candy.

18. A method to treat or prevent a skin condition in a subject in need thereof, comprising administering to the subject the confection of claim 1.

19. The method of claim 18 wherein the disease is contact dermatitis, alopecia areata, atopic dermatitis, seborrheic dermatitis, lichen planus, scleroderma, pemphigus, vitiligo, acne, rosacea, chronic venous ulcer, photosensitivity disorders, itch, cutaneous pain, disorders of impaired skin barrier function, psoriasis, disorders of oxidative stress in skin, radiation-induced skin damage, light-induced skin damage, oxidative skin damage, drug-induced photosensitivity, photosensitivity disorders, gut dysbiosis, photoaging, or aging.

20. The method of claim 18, wherein the confection is orally administered to the subject daily.
